(19) United States
(12) Patent Application Publication
(10) Pub. No.: US 2004/0047891 A1
(43) Pub. Date: Mar. 11, 2004

(54) Systems devices and methods for intrabody targeted delivery and reloading of therapeutic agents

(76) Inventors: Sabina Glozman, Rehovot (IL); Zur Pierre Beserman, Emek Sorek (IL); Yosi Morik, Chicago (IL)

Correspondence Address: G.E. Ehrlich, Anthony Castorina, 2001 Jefferson Davis Highway, Suite 207, Arlington, VA, 22202, US

(21) Appl. No.: 10/416,542

(22) PCT Filed: Feb. 26, 2002
(86) PCT No.: PCT/IL02/00149

(51) Int. Cl. A61F 2/00

(52) U.S. Cl. 424/423; 427/2.1

(57) ABSTRACT

A biomedical system for targeted delivery of a therapeutic agent to a tissue region of a subject is provided. The biomedical system comprises: (a) a biomedical device including: (i) a device body designed and configured for implantation within the tissue region of the subject; and (ii) a first member of a binding pair attached to a surface of said device body; and (b) a delivery vehicle including: (i) a carrier particle designed for carrying the therapeutic agent; (ii) a second member of said binding pair attached to said carrier particle, said second member of said binding pair being capable of specifically interacting with said first member of said binding pair thereby enabling targeting of said delivery vehicle to said biomedical device when implanted within said tissue region.

### FIELD AND BACKGROUND OF THE INVENTION

The present invention relates to systems, devices and methods for intrabody targeted delivery of molecules. More particularly, embodiments of the present invention relate to a reloadable drug delivery system, which enables targeted delivery of therapeutic agents to a tissue region of a subject, in a localized and timely manner.

Disease, injury and surgery can result in localized tissue damage and morbidity. For example, in coronary artery disease there is a narrowing or obstruction of the vessels that supply blood and oxygen to the heart. This narrowing is caused by fatty deposits (plaque) on the walls of the arteries, which gradually build up and obstructs the vessel, thereby markedly reducing the oxygen arriving to the heart muscle.

One of the most common non-surgical treatments for opening obstructed coronary arteries is Percutaneous Transluminal Coronary Angioplasty (PTCA), i.e., angioplasty, or “balloon treatment”. This procedure involves inserting a balloon catheter via a peripheral artery, and advancing it toward the heart up to the diseased artery. When the balloon is in place at the obstruction, it is inflated, thereby compressing the fatty deposits against the inner walls of the artery and “reshaping” the artery. This results in an opening of the artery and clearing of the obstruction. The drawback of this procedure is that in 30-50% of successful angioplasties, a blockage recurs later at the same site. This kind of recurrence is referred to as restenosis.

In order to solve the problem of restenosis, a procedure was developed whereby a small, slotted, stainless steel tube, referred to as a stent, is mounted on a balloon catheter, and introduced into the artery at the site of an obstruction recently cleared by angioplasty. When the balloon is inflated, the stent expands and is pressed against the inner walls of the artery. After the balloon is deflated and removed, the stent remains in place, acting as a scaffold to keep the artery open. The stent procedure is now very common, representing 70-90% of procedures in the treatment of coronary artery disease.

However, even with the stent procedure, reclosure of the artery within the stent (“in-stent restenosis”) is still a major problem, occurring with an incidence of between 22-32% (BENESTENT, STRESS studies). The pathogenesis of the human restenotic lesions after angioplasty is not well defined, but it seems to involve interaction of cytokines, growth factors, vascular and blood cellular elements, and the extent of injury. The restenosis process can be divided into several phases: thrombus formation, local inflammation, proliferation and matrix formation.

To date, most pharmacological attempts to reduce in-stent restenosis have focused on the reduction of thrombus formation, although drugs aiming at preventing cellular proliferation and migration have also been used systemically in conjunction with the angioplasty procedure in an attempt to reduce the rate of restenosis. For example, a clinical trial involving systemic administration of the antioxidant Probucol prior to and following angioplasty has produced promising results. However, immunological compromise, which often accompanies such systemic treatment modes, exposes the patient to surplus inflammation risks.

Other approaches being examined to prevent restenosis are ionizing radiation (intravascular brachytherapy) and ultrasound (intravascular sonotherapy). ³²P beta radiation has been shown in recent clinical trials (e.g. the INHIBIT trial) to decrease restenosis and reduce major acute coronary events nine months following stent placement, although the problem with the technique is that it does not appear to be “user friendly” enough to gain widespread acceptance.

Alternatively, the use of temporary stents produced from biodegradable and/or bioabsorbable materials significantly reduces the risk of tissue damage since they may be designed to have mechanical properties lacking stiffness and hardness, which usually contribute to the propensity of a stent to damage a vessel lumen. The use of biodegradable stents further eliminates the need for subsequent surgical procedures to remove stents following completion of therapeutic use. Nevertheless, biodegradable stents have some significant limitations: First, current biodegradable materials break down too quickly. This uncontrolled breakdown of a stent into large rigid fragments in the interior of a lumen, may cause obstruction to normal flow, such as voiding, thereby interfering with the primary purpose of the stent in providing lumen patency.

However, the wave of the future seems to be the use of materials to coat metal stents in a bid to reduce inherent stent thrombogenicity and thereby reduce the incidence of in-stent restenosis. An alternative route involves the use of biodegradable blood release polymer stents, the principle being gradual biological elimination without any residual implant stenosis, with the aim of effectively reducing the smooth muscle cell hyperplasia associated with prolonged exposure to non-degradable stents. However, interest in the use of biodegradable stents seems to have diminished in recent times at the expense of drug-coated stents.

The materials used to coat stents may be either synthetic (e.g. polyurethane, poly-L lactic acid) or naturally occurring substances (e.g. heparin, phosphorylcholine). In fact, the first drug-coated stent to receive both U.S. and European approval just arrived on the market in November 2000, when Cordis introduced the Bx Velocity™ “Hepacoat™” stent. This stent is coated with heparin, an antithrombotic compound that works by interfering with blood coagulation through a specific interaction with thrombin, a promoter of coagulation. Clinically, the drug is often administered intravenously and its effect persists with a half-life of up to several hours. However, because of steric effects, immobilized heparin is less susceptible to enzymatic degradation than is circulating heparin. Initial results with heparin-coated stents have been very promising in preventing restenosis.

In addition to heparin, other drugs have been coated onto stents with certain promising results. Some of these include trials with stents coated with the chemotherapeutic agents Taxol or Paclitaxel, with the immunosuppressive agent rapamycin (a naturally occurring macrocyclic lactone which acts as an inhibitor of neointimal hyperplasia), or with the anti-inflammatory/anti-proliferative glucocorticoid, dexamethasone.

Although the potential benefit expected from the use of medical devices capable of releasing pharmaceutical agents from their surfaces is significant numerous obstacles have hampered it's successful development; The requirement, in some instances, for long term release of bioactive agents; the need for a biocompatible, non-inflammatory device surface; the need for significant durability, particularly with devices that undergo flexion and/or expansion when being implanted or used in the body; concerns regarding processability, to enable the device to be manufactured in an economically viable and reproducible manner; and the requirement that the finished device be sterilizable using conventional methods.

U.S. Pat. No. 6,344,028 discloses a number of recent approaches for in-stent extended therapeutic treatment. These approaches involve re-implantation of stents either adjacently to a previously implanted stent or replacement of such. However, each insertion and extraction risks further damage to afflicted areas and damage to otherwise unaffected areas through which the instruments pass and can add to patient trauma. Moreover, insertion and withdrawal of additional instruments in sequence increases the time of the physician, staff, and medical facility, and the cost of multiple instruments.

There is thus a widely recognized need for, and it would be highly advantageous to have, systems, devices and methods for intrabody targeted delivery and reloading of therapeutic agents, devoid of the above limitations.

### SUMMARY OF THE INVENTION

According to one aspect of the present invention there is provided a biomedical system for targeted delivery of a therapeutic agent to a tissue region of a subject, the biomedical system comprising: (a) a biomedical device including: (i) a device body designed and configured for implantation within the tissue region of the subject; and (ii) a first member of a binding pair attached to a surface of the device body; and (b) a delivery vehicle including: (i) a carrier particle designed for carrying the therapeutic agent; (ii) a second member of the binding pair attached to the carrier particle, the second member of the binding pair being capable of specifically interacting with the first member of the binding pair thereby enabling targeting of the delivery vehicle to the biomedical device when implanted within the tissue region.

According to another aspect of the present invention there is provided a biomedical system for repeated targeting of a therapeutic agent to a tissue region of a subject, the biomedical system comprising: (a) a biomedical device including: (i) a device body designed and configured for implantation within the tissue region of the subject; and (ii) a first member of a binding pair attached to a surface of the device body; (b) a delivery vehicle including: (i) a carrier particle designed for carrying the therapeutic agent; (ii) a second member of the binding pair attached to a surface of the carrier particle, the second member of the binding pair being capable of specifically interacting with the first member of the binding pair; wherein the first and second members of the binding pair and the delivery vehicle are selected such that following targeting, the therapeutic agent is released from the delivery vehicle and the first member dissociates from the second member of the binding pair, thereby enabling repeated targeting of the therapeutic agent to the tissue region of the subject.

According to yet another aspect of the present invention there is provided a drug-reloadable biomedical implant comprising: (a) a device body designed and configured for implantation within a tissue region of a subject; (b) a first member of a binding pair attached to a surface of the device body, the first member of the binding pair being capable of interacting with a second member of the binding pair attached to a delivery vehicle carrying a therapeutic agent, thereby enabling targeting of the delivery vehicle to the biological implant; and (c) an effector moiety attached to the surface of the device body, the effector moiety being designed for activating release of a therapeutic agent from the delivery vehicle upon interaction between the first and the second members of the binding pair.

According to still another aspect of the present invention there is provided a method of delivering a therapeutic agent to a tissue region of a subject, the method comprising: (a) implanting in the tissue region of the subject a biomedical device including: (i) a device body; and (ii) a first member of a binding pair attached to a surface of the device body; (b) administering to the subject a delivery vehicle carrying the therapeutic agent, the delivery vehicle including: (i) a carrier particle designed for carrying the therapeutic agent; (ii) a second member of the binding pair attached to the carrier particle, the second member of the binding pair being capable of specifically interacting with the first member of the binding pair thereby enabling targeting of the delivery vehicle to the biomedical device and the tissue region.

According to further features in preferred embodiments of the invention described below, wherein the first and second members of the binding pair dissociate following release of the therapeutic agent, thereby enabling repeating step (b).

According to still further features in the described preferred embodiments the method further comprising repeating step (b) a predetermined number of times.

According to an additional aspect of the present invention there is provided a method of manufacturing a reloadable biomedical device the method comprising: (a) fabricating a device body designed and configured for implantation within a tissue region of a subject; (b) attaching to a surface of the device body a first member of a binding pair, the first member of the binding pair being capable of interacting with a second member of the binding pair attached to a delivery vehicle carrying a therapeutic agent, thereby enabling targeting of the delivery vehicle to the biological implant; and (c) attaching to the surface of the device body an effector moiety the effector moiety being designed for effecting release of the therapeutic agent from the delivery vehicle upon interaction between the first and the second members of the binding pair.

According to still further features in the described preferred embodiments, wherein the delivery vehicle is configured such that when the first member and the second member of the binding pair interact, the therapeutic agent carried by the delivery vehicle is released therefrom.

According to still further features in the described preferred embodiments wherein the biomedical device further includes an effector moiety attached to the surface of the device body, the effector moiety being designed for activating release of the therapeutic agent carried by the delivery vehicle when the first member and the second member of the binding pair interact.

According to still further features in the described preferred embodiments wherein the effector moiety forms a part of the first member of the binding pair.

According to still further features in the described preferred embodiments wherein the effector moiety is a chemical selected from the group consisting of a nonionic chemical, an anionic chemical, a cationic chemical, a natural occurring chemical and an amphoteric chemical

According to still further features in the described preferred embodiments wherein the effector moiety is an enzyme selected from the group consisting of a lipase and a peptidase.

According to still further features in the described preferred embodiments wherein the biomedical device is a stent.

According to still further features in the described preferred embodiments wherein the binding pair is selected from the group consisting of a biotin-avidin pair, a hapten-antigen pair, a lectin-carbohydrate pair and a ligand-receptor pair.

According to still further features in the described preferred embodiments wherein the carrier particle is selected from the group consisting of a liposome, a micelle, a cationic polymer and a cationic peptide.

The present invention successfully addresses the shortcomings of the presently known configurations by providing reloadable drug delivery systems, devices and methods, which enable targeted delivery of therapeutic agents to a tissue region of a subject, in a localized and timely manner.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates a general configuration of the biomedical system of the present invention.

FIG. 2 illustrates an embodiment of the biomedical system of the present invention, which utilizes an effector moiety for controlled release of a therapeutic agent from a targeted delivery vehicle.

FIG. 3 is a perspective view of one configuration of the biomedical device of the present invention.

FIG. 4 is a perspective view of a multi-layer coating configuration of the biomedical device of the present invention.

FIG. 5 is a perspective view of a dendrimer configuration of the biomedical device of the present invention.

FIGS. 6a-b illustrate in-vivo reloading of therapeutic agents onto an aorta implanted biomedical device of the present invention. FIG. 6a illustrates systemic administration of delivery vehicle of the present invention by injection. FIG. 6b is a schematic illustration of aorta located reloaded biomedical device.

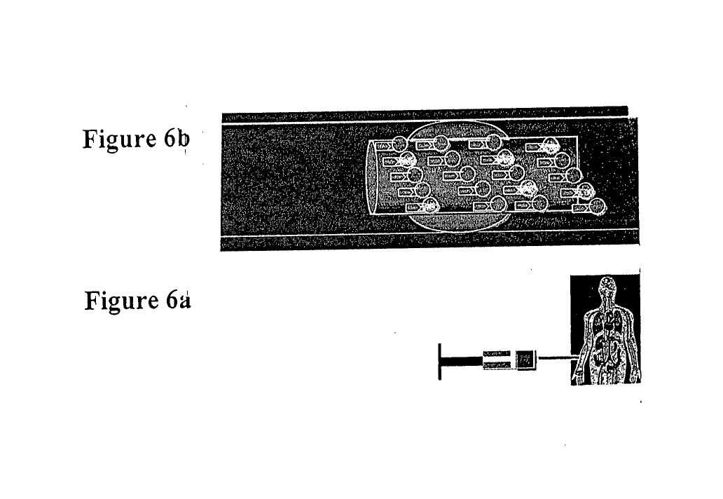

FIG. 7 is a schematic illustration of avidin binding to a biotin coated biomedical device located in a flow model system.

### DESCRIPTION OF THE PREFERRED EMBODIMENTS

The present invention is of systems, devices and methods for intrabody-targeted delivery of molecules, such as therapeutic agents. Specifically, the present invention relates to reloadable drug delivery systems and devices, which can be used to target release of therapeutic agents in a specific tissue region of a subject.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings described in the Examples section. The invention is capable of other embodiments or of being practiced or carried out in various ways. Also, it is to be understood that the phraseology and terminology employed herein is for the purpose of description and should not be regarded as limiting.

Many medical conditions are commonly treated by introducing an implantable medical device into a specific tissue of a patient. While necessary and beneficial for treating severe medical conditions, the placement of such devices in the body often gives rise to numerous complications. Some of these complications include: increased risk of infection; initiation of a foreign body response resulting in inflammation and fibrous encapsulation; and initiation of a wound healing response resulting in hyperplasia and restenosis. Thus, use of implanted medical devices can oftentimes lead to generation or intensification of problems that these devices were designed to treat.

A variety of devices and treatment approaches have been developed to prevent such complications. These include complementary treatments (i.e., chemo and radiotherapy), use of biodegradable devices for gradual biological elimination without residual implant stenosis and devices coated with bioactive materials such as immunosuppressive agents, anti-coagulants and/or anti-proliferative agents.

However, these methods suffer from several drawbacks including: the need for a durable biocompatible, non-inflammatory device surface, constant surgical involvement, concerns regarding processability, manufacturing costs and reproducibility and particularly the requirement for long term release of bioactive agents.

As described hereinunder and in the Examples section which follows, the present invention provides a novel biomedical system, which overcomes prior-art limitations by enabling targeted delivery and activation of therapeutic agents to a tissue region of a subject. Such a system is advantageous in cases where side effects accompany systematic pharmacological treatments, particularly where complex treatment is desired. Aside from targeted delivery the present invention enables reloading of therapeutic agents at designated time periods. This allows for long term targeting of bioactive agents without the need for surgical intervention and its accompanied risks. Finally, the universal nature of the biomedical system of the present invention, discards much, if not all, synthesis and testing operations associated with delivery of diverse drugs.

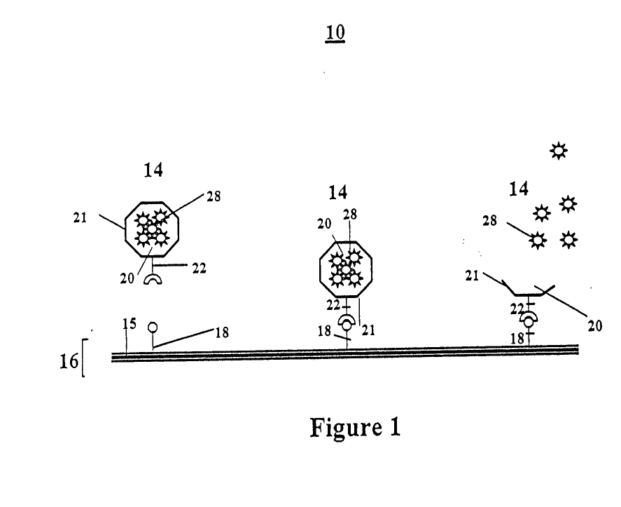

Referring now to the drawings, FIG. 1 illustrates a biomedical system, for targeted delivery of therapeutic agents to a subject's tissue region and which is referred to herein as system 10.

System 10 of the present invention includes a biomedical device 12, which is designed and configured for implantation (i.e., long-term or transient implantation) within a tissue region of a subject.

Biomedical device 12 includes a device body 16 which is shaped, sized and fabricated for implantation into a particular tissue region. Device body 16 can be configured as a graft, a chip/patch, beads magnetic particles, a medical device and the like, depending on the intended use and size of implantation site (further description of device body 16 is provided hereinunder).

As used herein a “subject” refers to a mammal such as a canine, a feline, an ovine, a porcine, an equine, or a bovine; preferably the term “subject” refers to a human.

A “tissue region” refers to any tissue such as a vascular system, esophagus, trachea, colon, billiary ducts, urethra and ureters. The tissue region according to the present invention may be normal, neoplastic, hyperplastic, necrotic and the like.

Biomedical device 12 further includes a first member 18 of a binding pair, which is preferably attached to a surface 15 of device body 16. Attachment can be through either non-covalent (i.e., electrostatic) or covalent interactions, depending on the composition of surface 15, and the desired binding reversibility.

System 10 further includes a delivery vehicle 14. Delivery vehicle 14 is designed and configured for delivering/targeting therapeutic agents to a tissue-implanted biomedical device 12. Delivery vehicle 14 is typically administered into the body of the patient independent of biomedical device 12. Administration of delivery vehicle 14 can be effected via, for example, systemic injection.

Delivery vehicle 14 includes a carrier particle 20 designed for carrying a therapeutic agent 28 and a second member 22 of the binding pair. Second member 22 is preferably attached to a surface 21 or forms a part of carrier particle 20.

Delivery vehicle 14 is preferably selected such that an activity of a therapeutic agent 28 carried thereby is masked, thus maintaining therapeutic agent 28 inactive until release from Delivery vehicle 14.

Thus, carrier particle 20 can be any vesicle, polymeric shell and the like, capable of carrying (e.g., encapsulating) therapeutic agent 28. Carrier particle 20 can also be a prosthetic group, which is cleaved off at the target, thus converting a prodrug into a drug. The prosthetic group can double as second member 22 of the binding pair. Further description of carrier particles suitable for use with the present invention is provided hereinbelow and Example sections, which follow.

Therapeutic agent 28 can be any bioactive or biopharmaceutical molecule, including but not limited to, anti-proliferation or thrombolytic agents, anti-aggregants, anti-coagulants, anti-inflammatory compounds, vasoactive compounds, hormones, growth factors, nucleotides, antioxidants, enzymes, bioactive peptides, lipids, carbohydrates, proteins, receptor ligands, neurotransmitters, chemotherapeutics, radioisotopes/radionuclides, anti-neoplastics, anti-angiogenetics, selecting, signaling molecules, anti-infectious drugs, neuroprotective or immunoactive agents, anti-tumor agents, toxins, nucleic acids, antisense oligonucleotides, amino acid groups, adhesive molecules, cells and the like. Examples of possible therapeutic agents are disclosed in U.S. Pat. No. 6,280,411, which is fully incorporated herein.

The “binding pair” as used herein refers to a complementary pair of molecules, designated herein as first member and second member, which exhibit high affinity (i.e., K_{D}<10⁻⁶) to each other and as such are capable of specifically interacting. The designation first member and second member is flexible and the same molecule can be either. Typical binding pairs include biotin-avidin; hapten-antigen; lectin-carbohydrate; ligand-receptor, enzyme-substrate, nucleic acid-complementary polynucleotide, and derivatives thereof. Further description of the binding pair is provided hereinunder.

The binding pair is selected such that a specific interaction between first member 18 and second member 22 of the binding pair enables targeting of delivery vehicle 14 to biomedical device 12 when implanted within a tissue region.

Since therapeutic agent 28 is preferably inactive (pharmacologically) when carried by delivery vehicle, targeting of delivery vehicle 14 to biomedical device 12 must be followed by release of therapeutic agent 28 from delivery vehicle 14.

Such release may be effected via passive, conditional or active release mechanisms.

Passive release depends primarily on the pharmaceutical formulation of carrier particle 20 and solubility of therapeutic agent 28. For example, carrier particle 20 can be designed to have a plasma half life, that enables disintegration of carrier particle 20 only following targeting to biomedical device 12. It will be appreciated in this case that selection of appropriate plasma half life depends on the site of implantation of biomedical device 12, the site of administration of delivery vehicle 14 and dynamics of vascular flow.

Disintergration can also be intiated/accelarated by interaction between members 18 and 22 which distablizes the structure of carrier particle 20.

Conditional release can be triggered by physical and/or biochemical conditions at implantation site. Examples include pH conditions, osmotic forces, redox conditions and the like.

Active release can be effected by several factors including radiation (e.g., ultraviolet radiation), temperature, photo-triggering, systemic administration of triggering substances and the like.

Active release can also be effected by an effector moiety 30, which is preferably attached to surface 15 of device body 16 and may form a part of first member 18 of the binding pair.

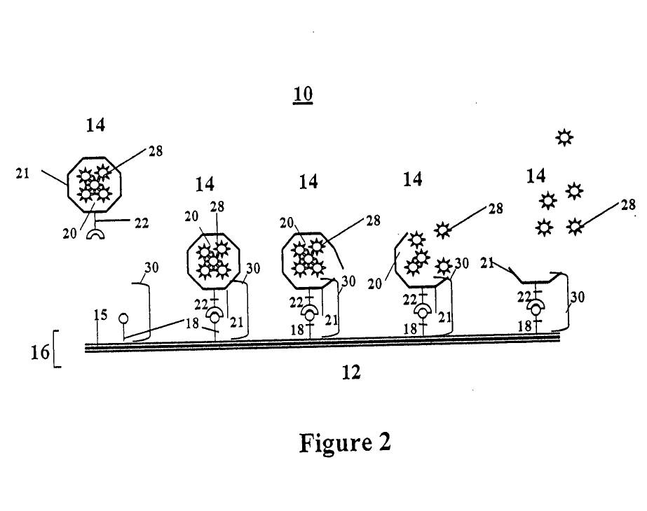

Effector moiety 30 is preferably configured for activating release of therapeutic agent 28 following binding between first and second members of the binding pair (FIG. 2).

Examples of effector moiety 30 include but are not limited to enzymes, nonionic chemicals, anionic chemicals, cationic chemicals, amphoteric chemicals and natural occurring chemicals (further description of effector moiety 30 is provided hereinunder).

Thus, the present invention provides a universal drug delivery system, which can be used to deliver any therapeutic agent to specific tissue regions without having to modify the therapeutic agent thus traversing the need for synthesis and testing operations, which are otherwise necessary for consistent, reliable and risk-free targeted delivery of diverse therapeutic agents. Furthermore, by timing and localizing release/activation of therapeutic agents, the biomedical systems of the present invention traverses problems which are inherent to activity of a therapeutic agent at tissue regions which are not to be treated.

As described hereinabove, device body 16 is designed and configured for implantation within a tissue region of a subject. Thus, device body 16 can be designed and configured to be used as a catheter, a wire guide, a cannula, a stent, a vascular or other graft, a cardiac pacemaker lead or lead trap, a cardiac defibrillator lead or lead tip, a heart valve or an orthopedic device, appliance, implant or replacement. Device body 16 can also be configured as a combination or a portion of these devices.

Physical dimensions of device body 16 are selected according to the target tissue. For example aortic, esophageal, tracheal, and colonic stents may have dimensions of about 25 mm in width/diameter and lengths of about 100 mm or even longer.

Device body 16 can be composed of a base material 24 suitable for the intended use of system 10. Base material 24 is preferably biocompatible, although it will be appreciated that cytotoxic or other incompatible materials may also be employed as long as measures are taken to insulate such materials from intrabody exposure.

A variety of conventional materials can be employed as base material 24. These include biocompatible metals such as stainless steel, tantalum, titanium, nitinol, gold, platinum, inconel, iridium, silver, tungsten, or alloys thereof; carbon or carbon fibers; cellulose acetate, cellulose nitrate, silicone, polyethylene, teraphthalate, polyurethane, polyamide, polyester, polyorthoester, polyanhydride, polyether sulfone, polycarbonate, polypropylene, high molecular weight polyethylene, polytetrafluoroethylene, or another biocompatible polymeric material or mixtures or co-polymers of these; polylactic acid, polyglycolic acid or co-polymers thereof, a polyanhydride, polycaprolactone, polyhydroxybutyrate valerate or another biodegradable polymer or mixtures or co-polymers of these; a protein, an extra-cellular matrix component, collagen, fibrin or suitable mixtures thereof.

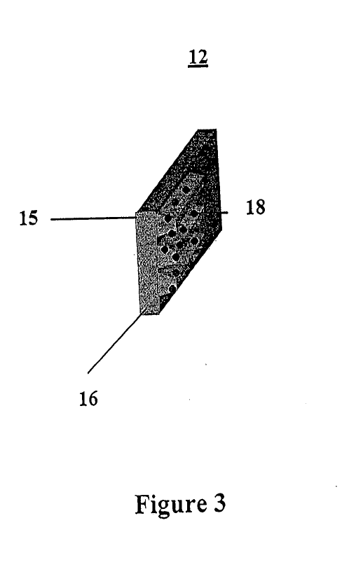

As illustrated in FIG. 3, device body 16 preferably further includes a coating layer 26, placed on base material 24. Coating 26 serves as a surface for attaching first member 18. It will be appreciated that coating layer 26 is made of a biocompatible material, which is non-immunogenic. Coating layer 26 may be flat/smooth, or preferably, may be made such that the surface is grooved/ridged/ribbed in order to increase the surface area for attachment of first member 18.

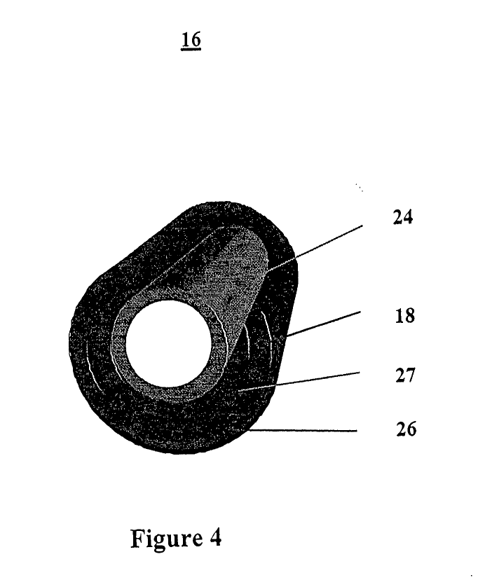

As is illustrated in FIG. 4, device body 16 can include a plurality of coating layers 27, where in addition to the primary coating 27, additional coatings preferably of a biodegradable material can be added (see FIG. 4).

The biodegradable coating material used is selected based upon its clearance rate and toxicity of degradation products. For example, high molecular weight biomaterials can be used when tumor targets or clot target sites are involved. High molecular weight hydrophilic polymers, triblock polymers, hyaluronic acid, and albumin demonstrate non-toxic post-degradation characteristics. The biodegradable coating material can be a lubricant, and/or a hydrophylic (albumin, triblock polymer, hyaluronic acid, heparin, PEOs, PEGs, polyurethanes, etc., or mixtures thereof), and/or natural (gelatin, fibrin, fibrinogen, collagen, fibronectin, etc., or mixtures thereof) or synthetic (silica-based) hydrophobic adhesive biomaterial, and/or a lipid-based biomaterial (phospholipids, lipid extracts, triglyceride films, polymers of fatty acids, waxes, sphyngolipids, sterols, glycolipids, etc., or mixtures of thereof).

Metallic clusters or colloids, such as colloidal gold may also be used as coating (see FIG. 3). Colloidal gold, to which first member 18 can be covalently attached, has a number of advantages for loading/reloading purposes. Such a configuration enables: (a) enhanced stability; (b) attachment of multiple molecules of first member 18 to each colloid, thereby improving binding pair reactivity of the conjugate and yielding better targeting; in addition, having multiple biomolecules of first member 18 per gold particle provide the possibility of reloading at a number of designated time periods; (d) to vary the number of organic moieties that can be associated with the metallic moiety/moieties of the organometallic colloids (further description provided hereinunder).

Various techniques may be employed for coating device body 16 including, but not limited to, plasma deposition and vapor phase deposition. Vapor phase is preferably used to deposit parylene and parylene derivative coatings. Currently available vapor phase deposition systems include Specialty Coating Systems™ (100 Deposition Drive, Clear Lake, Wis. 54005), Para Tech Coating™, Inc. (35 Argonaut, Aliso Viejo, Calif. 92656) and Advanced Surface Technology™, Inc. (9 Linnel Circle, Billerica, Mass. 01821-3902). Plasma may be used to deposit polymers such as poly(ethylene oxide), poly(ethylene glycol), and poly(propylene oxide), as well as polymers of silicone, methane, tetrafluoroethylene (including TEFLON brand polymers), tetramethyldisiloxane, and others.

Other coating techniques include dipping, spraying, and the like (see U.S. Pat. No. 5,873,904 for further detail).

As described hereinabove, the binding pair may be any complementary pair of molecules that demonstrate specific binding, preferably reversible binding. Examples for specific reversible binding pairs include the endothelin-A (ET-A) or B (ET-B) receptor and their synthetic ligands, such as ABT-627, which dissociates from the ET-A receptor within 2 hours. Proteins corresponding to known cell surface receptors (including low density lipoproteins, transferrin, and insulin), fibrinolytic enzymes, and biological response modifiers (including interleukin, interferon, erythropoietin, and colony-stimulating factor) are also preferred binding pairs. Oligonucleotides, i.e., antisense oligonucleotides that are complementary to portions of target cell nucleic acids (DNA or RNA), are also useful as targeting moieties in the practice of the present invention. Oligonucleotides binding to cell surfaces are also useful.

Analogs of the above-listed binding pairs may also be used within this configuration of the present invention. For example, neutravidin, a deglycosylated form of avidin, which is designed to bind lectins at reduced to undetectable levels is characterized by very low non-specific binding and cross-reactivity. Table 1 below, illustrates several biochemical properties of avidin derivatives which may be used within the context of the present invention.

TABLE 1

| Feature | Avidin | Sterptavidin | NeutrAvidin |
|---|---|---|---|
| Source | Chicken egg wight | Fermentation | Modified avidin |
| Carbohydrates | yes | no | No |
| Molecular weight | 67,000 | 60,000 | 60,000 |
| No. of residues | 128 | 156 | 128 |
| (per unit) | | | |
| Isoelectric point | 10 | 5-6 | 6.3 |
| Conserved | 70-71 | 79-80 | 70-71 |
| Trp/Lys | | | |
| sequences | | | |
| Position | 110-111 | 120-121 | 110-111 |

In addition, synthetic or composite binding pairs may be designed and used with the present invention. Since avidin may evoke an immunogenic response at high concentrations in the blood, PEGylation can be used greatly reduce its immunogenicity, without affecting its binding to biotin [Chinol M et al. (1998) Biochemical modifications of avidin improve pharmacokinetics and biodistribution, and reduce immunogenicity. Br J Cancer. 78(2):189-97].

Human monoclonal antibodies or “humanized” murine antibodies are also useful as binding members in accordance with the present invention.

Preferably, the binding pair utilized by the present invention includes non-protein macromolecules, since such molecules exhibit reduced in-vivo degradation and minimal cross-reactivity with other tissues in the body. For example, avidin and its derivatives may evoke an immunogenic response at high concentrations in the blood. Moreover, avidin can associate with plasma-resident immunoglobulins and other proteins, as well as with endogenous biotin. Consequently, where avidin to be used as first member 18 of the binding pair (associated with the coated implant), the binding sites on coating layer 26 may become saturated prior to loading/reloading. Thus, in such a case it is preferred that biotin is utilized as first member 18 of the binding pair since in this context it will not undergo saturation by endogenous substances prior to loading/reloading.

Attachment of first member 18 to surface 15 of device body 16, can be effected by any direct or indirect conjugation method, which is selected primarily according to the nature of the substrate to be coated. It will be appreciated though, that direct binding is less preferred due to decreased in-vivo mobility (i.e., steric hindrance) and decreased number of functional binding sites.

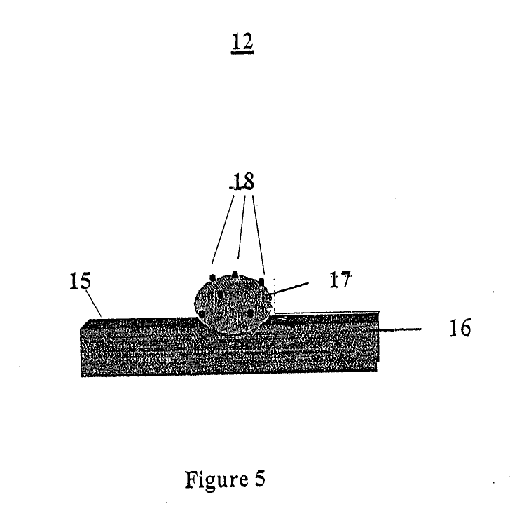

Generally, metal particles can bind organic moieties through either non-covalent (i.e., electrostatic) or covalent interaction. Non-covalent binding is preferably used when low binding of an organic moiety per metal molecule is desired. A preferred binding method according to the present invention utilizes a linker 17 such as a dendritic polymer (“dendrimer”), which is either directly associated with the implant or associated with the primary coating layer of the implant. Such binding method increases the surface area on the implant available for binding the organic moiety (see FIG. 5).

U.S. Pat. No. 5,728,590, describes covalent binding methods of organic moieties to metallic clusters or colloids which can be used with the present invention. The process involves synthesis of the metal colloid (For example, HauCl₄ (0.01%) in 0.05M sodium hydrogen maleate buffer (pH 6.0), with 0.004% tannic acid.) in the presence of a suitable polymer. The polymer may be chosen from a linear or branched group with functional groups attached, such as polyamino acids, polyethylene derivatives, other polymers, or mixtures thereof. A second method is to synthesize the metal particle first, e.g., by combining 0.01% HauCl₄ with 1% sodium citrate with heating. Once gold colloid of the desired size is formed, it is coated with a polymer by mixing the two together and optionally warming to 60-100° C. for several minutes. The polymer coating may be further stabilized by (i) microwave heating, (ii) further chemical crosslinking, e.g., by glutaraldehyde or other linkers, or by continued polymerization adding substrate molecules for a brief period. N,N′-methylene bis acrylamide, can be used to covalently stabilize the polymer coating. Photocrosslinking may also be used.

Once formed, the functionalized polymer coating can be used to support attached proteins, peptides, antibodies, lipids, carbohydrates, nucleic acids and the like.

It will be appreciated that the synthesis method described hereinabove is advantageous, since coupling may be done mildly, in physiological buffers if desired, using standard crosslinking technology. This eliminates the usual restriction that conjugation must be performed in very low ionic strength buffers, which precludes attachment of certain molecules such as IgMs, which cannot withstand the low ionic strength requirement.

Conjugation of molecules to polymeric substrates can be effected via any approach well known in the art. U.S. Pat. No. 6,338,904 provides a comprehensive description of suitable approaches.

The following section provides detail of several approaches, which can be used by the present invention to conjugate molecules to polymeric substrates.

(i) Binding through a chemical linking moiety. The chemical linking moiety has a structure represented by: A-X-B, wherein A is a photochemically reactive group, B is a reactive group which responds to a different stimulus than A and X is a non-interfering skeletal moiety, such as a C₁-C₁₀ alkyl. Covalent binding of the organic substance to the surface of the medical device is effected via the linking moiety.

(ii) Covalent binding to an amine-rich material, (e.g., a polyurethaneurea) modified with hydrophobic groups (U.S. Pat. No. 4,720,512).

(iii) Ionic binding via a quaternary ammonium compound. See U.S. Pat. Nos. 4,229,838, 4,613,517, 4,678, 660, 4,713,402, and 5,451,424 for details.

(iv) covalent binding through a hydrophilic spacer reacted with one or more of a reactive functional group overhanging from a polymer backbone (U.S. Pat. No. 6,338,904).

As described hereinabove system 10 further includes a carrier particle 20 designed for carrying therapeutic agent 28.

Nanoparticles or nanospheres can also be used as carrier particle 20. Nanoparticles or nanospheres include, but are not limited to, poly(ethylene oxide), poly(L-lactic acid) or poly(b-benzyl-L-aspartate) (e.g. as vehicles for delivery of anti-inflammatory and anti-tumor drugs); poly(lactide-co-glycolide)-[(propylene oxide)-poly(ethylene oxide)]; polyphosphazene derivatives (which are 100-120 nm in diameter and long-circulating in the blood); azidothymidine (AZT) or dideoxycytidine (DDC) nanoparticles; poly(isobutylcyanoacrylate) nanocapsules (e.g. for intragastric administration of insulin in diabetes); poly(g-benzyl-L-glutamate) or poly(ethylene oxide); chitosan-poly(ethylene oxide) nanoparticles (which have great protein loading capacity, with an entrapment efficiency of up to 80% of the protein, and which provide a continuous release of the entrapped protein for up to 1 week); methotrexane-o-carboxymethylate chitosan (o-CMC); or solid lipid nanoparticles (SLNs).

Microcapsules or microspheres can be used as carrier particle 20. Microcapsules or microspheres include, but are not limited to, multiporous beads of chitosan (e.g. as a carrier for the chemotherapeutic adriamycin); coated alginate microspheres; N-(aminoalkyl) chitosan microspheres; chitosan/calcium alginate beads; poly(adipic anhydrate) microspheres (e.g. for ocular drug delivery); gellan-gum beads; poly(D, L-lactide-co-glycolide) microspheres (e.g. in radiotherapy for treatment of infiltrating brain tumors); alginate-poly-L-lysine microcapsules (e.g. for insulin-dependent diabetes); chitosan/gelatin microspheres (e.g. for controlled release of cimetidine); crosslinked chitosan network beads with spacer groups; 1,5-diozepan-2-one (DXO) and D,L-dilactide (D,L-LA) microspheres; triglyceride lipospheres (liposomes); glutamate and TRH microspheres (e.g. for delivery and long-term release of neurotransmitters within the CNS); polyelectrolyte complexes of sodium alginate chitosan; polypeptide microcapsules; or albumin microspheres (a number of studies have shown that albumin accumulates in solid tumors, thus making it a potential macromolecular carrier for the site-directed delivery of antitumor drugs).

Carrier particle 20 can also be a lipid vesicle (e.g., liposome). As used herein, the phrase “Lipid-vesicle” refers to any vesicle composed of “amphipathic vesicle-forming lipids”, which include any amphipathic lipid having hydrophobic and polar head group moieties, and which spontaneously forms into bilayer vesicles in water, as exemplified by phospholipids (e.g., cholesterol and cholesterol derivatives such as cholesterol sulfate and cholesterol hemisuccinate), or is stably incorporated into lipid bilayers in combination with phospholipids with its hydrophobic moiety in contact with the interior, hydrophobic region of the bilayer membrane, and its polar head group moiety oriented toward the exterior, polar surface of the membrane.

A variety of methods are available for preparing liposomes as described in, e.g., Szoka et al., Ann. Rev. Biophys. Bioeng. 9:467 (1980), U.S. Pat. Nos. 4,186,183, 4,217,344, 4,235,871, 4,261,975, 4,485,054, 4,501,728, 4,774,085, 4,837,028, 4,946,787, PCT Publication No. WO 91/17424, Szoka & Papahadjopoulos, Proc. Natl. Acad. Sci. USA, 75: 4194-4198 (1978), Deamer and Bangham, Biochim. Biophys. Acta, 443: 629-634 (1976); Fraley, et al., Proc. Natl Acad. Sci. USA, 76: 3348-3352 (1979); Hope, et al., Biochim. Biophys. Acta, 812: 55-65 (1985); Mayer, et al., Biochim. Biophys. Acta, 858: 161-168 (1986); Williams, et al., Proc. Natl. Acad. Sci., 85: 242-246 (1988), the text Liposomes, Marc J. Ostro, ed., Marcel Dekker, Inc., New York, 1983, Chapter 1, and Hope, et al., Chem. Phys. Lip. 40: 89 (1986), all of which are incorporated herein by reference.

Suitable methods include, e.g., sonication, extrusion, high pressure/homogenization, microfluidization, detergent dialysis, calcium-induced fusion of small liposome vesicles, and ether-infusion methods, all well known in the art.

The multilamellar vesicle formation method described in the prior art which produces lipid vesicles of heterogeneous sizes can also be used by the present invention. In this method, the vesicle-forming lipids are dissolved in a suitable organic solvent or solvent system and dried under vacuum or an inert gas to form a thin lipid film. If desired, the film may be redissolved in a suitable solvent, such as tertiary butanol, and then lyophilized to form a more homogeneous lipid mixture which is in a more easily hydrated powder-like form. This film is covered with an aqueous buffered solution and allowed to hydrate, typically over a 15-60 minute period with agitation. The size distribution of the resulting multilamellar vesicles can be shifted toward smaller sizes by hydrating the lipids under more vigorous agitation conditions or by adding solubilizing detergents such as deoxycholate.

Unilamellar vesicles are generally prepared by sonication or extrusion. Sonication is generally performed with a tip sonifier, such as a Branson tip sonifier, in an ice bath. Typically, the suspension is subjected to several sonication cycles. Extrusion may be carried out by biomembrane extruders, such as the Lipex Biomembrane Extruder. Defined pore size in the extrusion filters may generate unilamellar liposomal vesicles of specific sizes. The liposomes may also be formed by extrusion through an asymmetric ceramic filter, such as a Ceraflow Microfilter, commercially available from the Norton Company, Worcester Mass.

Following liposome preparation, the liposomes which have not been sized during formation may be sized to achieve a desired size range and relatively narrow distribution of liposome sizes. A size range of about 0.2-0.4 microns allows the liposome suspension to be sterilized by filtration through a conventional filter, typically a 0.22 micron filter. The filter sterilization method can be carried out on a high throughput basis if the liposomes have been sized down to about 0.2-0.4 microns.

Several sizing techniques can be used for generating liposomes of a desired size (see, for example, U.S. Pat. No. 4,529,561 or 4,737,323).

Liposome sizing can be achieved by sonicating a liposome suspension either by bath or probe sonication produces a progressive size reduction down to small unilamellar vesicles less than about 0.05 microns in size. Homogenization is another method, which relies on shearing energy to fragment large liposomes into smaller ones. In a typical homogenization procedure, multilamellar vesicles are recirculated through a standard emulsion homogenizer until selected liposome sizes, typically between about 0.1 and 0.5 microns, are observed. The size of the liposomal vesicles may be determined by quasi-electric light scattering (QELS) as described in Bloomfield, Ann. Rev. Biophys. Bioeng., 10: 421-450 (1981), incorporated herein by reference. Average liposome diameter may be reduced by sonication of formed liposomes. Intermittent sonication cycles may be alternated with QELS assessment to guide efficient liposome synthesis.

Extrusion of liposome through a small-pore polycarbonate membrane or an asymmetric ceramic membrane is also an effective method for reducing liposome sizes to a relatively well-defined size distribution. Typically, the suspension is cycled through the membrane one or more times until the desired liposome size distribution is achieved. The liposomes may be extruded through successively smaller-pore membranes, to achieve a gradual reduction in liposome size. For use in the present invention, liposomes having a size of about 0.05 microns to about 0.15 microns. More preferred are liposomes having a size of about 0.05 to 0.5 microns.

Lipid vesicles utilized as carrier particle 20 of the present invention preferably further include hydrophilic polymers, which are known to increase vesicle stability, thereby preventing untimely release of therapeutic agent 28 from carrier particle 20.

Thus, hydrophilic polymers such as polyethylene glycol (PEG)-modified lipids or ganglioside G_{M1} can be added to liposomes used as carrier particle 20. Furthermore, addition of such components prevents vesicle aggregation during coupling of second member 22 to the vesicle. A concentration of hydrophilic polymer (e.g., PEG) of 1 to 4 mole percent of vesicle-forming lipid (excluding cholesterol) provides optimal blood half-life (U.S. Pat. No. 6,214,338). Methods of preparation of PEG for incorporation into liposomes are disclosed in Klibanov et al., FEBS Lett., 268: 235-237 (1990). It will be appreciated that therapeutic agent 28 may be PEGylated as well so as to reduce agent's immunogenicity and increase half-life time in circulation.

Attachment of second member 22 of the binding pair to carrier particle 20 can be effected by any method known in the art [see for example U.S. Pat. No. 5,776,487 and Example 1 of the Examples section which follows). For example, biotin can be attached to a surface of a liposome using biotinylated phospholipids [biotinylated dipalmitoylphosphatidyl ethanolamine (DPPE)], which are commercially available from Pierce Chemicals, Rockford, Ill.

Alternatively, biotinylated phospholipids can be manufactured using the methods disclosed by Rivnay, et al. (see “Use of Avidin-Biotin Technology for Liposome Targeting,” in Methods in Enzymology, Vol. 149, pgs. 119-123, 1987). Briefly, the phospholipid is dissolved in a solution of chloroform-methanol containing biotinyl N-hydroxysuccinimide ester (BNHS), followed by the addition of a chloroform solution containing 15% (v/v) triethylamine. The reaction proceeds for about two hours at room temperature and then the mixture is stored at about −70 ° C. Purification is performed using gradient high-performance liquid chromatography. The column is first washed with a solvent mixture containing n-hexane/2-propanol/water (60:80:14, v/v/v) until a steady baseline is established followed by the introduction of a different solvent mixture containing n-hexane/2-propanol/water (60:80:7, v/v/v) until a new baseline of about 0.07 optical density (OD) units above the first baseline is established. Then the lipid sample is applied and the elution monitored with a M-441 discrete-wavelength ultraviolet detector (214 nm). The column is then eluted with the solvent solutions described above, 5 minutes with the second solution followed by a 20-minute linear gradient between 0% and 100% of the first solvent solution in the second. Further elution in the first solvent for 45-70 minutes is performed to achieve a stable baseline. The peaks are collected, the eluted material pooled, and the solvent evaporated under a stream of nitrogen.

Liposome-peptide conjugates can be prepared by forming an amide bond between the amino group of a phosphatidylethanolamine and the carboxy terminus of an amino acid sequence of the peptide. Briefly, a peptide of interest (i.e., second member 22) is prepared as an anhydride; a phosphatidylethanolamine such as DOPE is then reacted with the anhydride in the presence of suitable reagents, such as triethylamine (for further description see, U.S. Pat. No. 6,339,069).

Alternatively, liposome-peptide conjugation can be effected by reacting a protein with a maleimide derivatized lipid such as maleimide derivatized phosphatidylethanolamine (M-PE) or dipalmitoylethanolamine (M-DEP). This approach is described in detail by Martin et al. J. Biol. Chem., 257: 286-288 (1982).

Whole liposome labeling method is disclosed in U.S. Pat. No. 5,540,935. Other liposome conjugation methods are disclosed in G. Gregoriadis, (1984) “Liposome Technology” CRC Press, Boca Raton, Fla. and D. D. Lasic, “Liposomes: from physics to applications” (1993) Elsevier, Amsterdam; N.Y. each of which is fully incorporated herein by reference.

Loading of therapeutic agent 28 into carrier particle 20, may be effected using any conventional method known in the art. The most common methods include encapsulation techniques and transmembrane potential loading methods. In the encapsulation technique, the therapeutic agent is placed into the buffer from which the liposomes are made. The latter method has been described in detail in U.S. Pat. Nos. 4,885,172, 5,059,421, and 5,171,578.

The transmembrane potential loading method can be used with essentially any conventional therapeutic agent, which can exist in a charged state when dissolved in an appropriate aqueous medium. Preferably, this technique is applied to a therapeutic agent, which is relatively lipophilic such that it partitions into the liposome membranes. A transmembrane potential is created across the bilayers of the liposomes or targeting moiety liposome conjugates and the therapeutic agent is loaded into the liposome by means of the transmembrane potential. The transmembrane potential is generated by creating a concentration gradient for one or more charged species (e.g., Na⁺, K⁺ and/or H⁺) across the membranes. This concentration gradient is generated by producing vessicles having different internal and external media. Thus, for a therapeutic agent which is positively charged when ionized, a transmembrane potential is created across the membranes which has an inside potential which is negative relative to the outside potential, while for a therapeutic agent which is negatively charged, the opposite orientation is used.

As described hereinabove, one embodiment of system 10 includes an effector moiety for activating release of therapeutic agent 28 from a targeted delivery vehicle.

FIG. 2 specifically illustrates activation of release of therapeutic agent 28 from a targeted delivery vehicle 14 using effector moiety 30.

To activate such release effector moiety 30 is selected capable of disrupting the structure of carrier particle 20. Effector moiety 30 can be selected from the group of chemicals and enzymes, dependent upon the composition of carrier particle 20. For example effector moiety 30 can be a liposomal lytic agent. Examples of liposomal lytic agents include but are not limited to non-ionic chemicals such as polyoxyethylene alkyl ethers such as polyoxyethylene cetyl ether, polyoxyethylene lauryl ether, etc.; polyoxyethylene alkylphenyl ethers such as polyoxyethylene octylphenyl ether, etc.; polyoxyethylene alkyl esters such as polyoxyethylene sorbitan monooleate, polyoxyethylene sorbitan monopalmitate, polyoxyethylene sorbitan monostearate, polyoxyethylene sorbitan trioleate, etc.; methylglucanide derivatives such as octanoyl-N-methylglucamide, nonanoyl-N-methylglucamide, decanoyl-N-methylglucamide, etc.; and alkyl sugar derivatives such as n-octyl-.beta.-D-glucoside, etc. Anionic chemicals, for example, sodium dodecyl sulfate (SDS), laurylbenzenesulfonic acid, deoxychloric acid, cholic acid, tris(hydroxymethyl)aminomethane dodecylsulfite (Tris DS), etc. Cationic surfactants, for example, alkylamine salts such as octadecylamine acetic acid salt, tetradecylamine acetic acid salt, stearylamine acetic acid salt, laurylamine acetic acid salt, lauryldiethanolamine acetic acid salt, etc.; quaternary ammonium salts such as octadecyltrimethylammonium chloride, dodecyltrimethylammonium chloride, cetyltrimethylammonium chloride, cetyltrimethylammonium bromide, lauryltrimethylammonium chloride, allyltrimethylammonium methylsulfate, benzalkonium chloride, tetradecyldimethylbenzylammonium chloride, octadecyldimethylbenzylammonium chloride, lauryldimethylbenzylammonium chloride, etc.; and alkylpyridinium salts such as laurylpyridinium chloride, stearylamidomethylpyridinium chloride, etc. Amphoteric chemicals, for example, 3-(3-cholamidoamidopropyl)dimethylammonio-1-propane sulfonate, 3-(3-cholamidoamidopropyl)dimethylammonio-2-hydroxy-1-propane sulfonate, etc. Natural occurring chemiclas, for example, saponin (derived from soybeen), digitonin, etc.

Alternatively, effector moiety 30 can be an enzyme (i.e., lipase, peptidase), capable of digesting carrier particle 20. For example, peptide-lipid (e.g., phosphatidylethanolamine) conjugates may be incorporated into stable liposomes. Once the peptide portion of the conjugate is cleaved from the lipid, by the action of peptidases, the liposomes disintegrate, thereby releasing their contents in the vicinity of the target location as disclosed in U.S. Pat. No. 6,087,325 which is fully incorporated herein.

Examples for suitable peptidases include, but not limited to: matrix metalloproteinases, serine proteases, cysteine proteases, elastase, plasmin, plasminogen activator, stromelysin, human collagenases, cathepsins, lysozyme, granzymes, dipeptidyl peptidases, peptide hormone-inactivating enzymes, kininases, bacterial peptidases and viral proteases. It will be appreciated that using bacterial and/or viral enzymes is preferable to avoid digestion of endogenous substrates.

Alternatively, effector moiety 30 can be an enzyme diverting therapeutic agent 28 prodrug form to an active drug by cleavage of carrier particle 20 (i.e., at least a portion of which serving as a prosthetic group), which may form at least part of second member 22. For example, polylysine derivatives containing specific protease cleavage sites may serve as carrier particle 20. An example of such a derivative is (Lys)ₙ-Phe-Pro-Arg, where “(Lys)ₙ” represents polylysine; “Phe” refers to phenylalanine; “Pro” refers to proline, and “Arg” refers to arginine. This polylysine derivative may be cleaved by the serine proteinase thrombin which in this case will form effector moiety 30. Other examples

System 10 of the present invention can be universally applied for treating a variety of medical conditions in a subject, including, for example, treatment of solid malignancies.

System 10 can also be used to treat tissue damage following medical procedures, including, for example, treatment of abrupt vessel reclosure post PCTA, the “patching” of significant vessel dissection, the sealing of vessel wall “flaps” either secondary to catheter injury or spontaneously occurring, or the sealing of aneurysmal coronary dilations associated with various arteritidies. Likewise.

System 10 can also be used to provide a complementary pharmaceutical treatment which can be used in the treatment of solid tumor malignancies.

The term “treating” refers to alleviating or diminishing a symptom associated with a disease or a condition. Preferably, treating cures, e.g., substantially eliminates, and/or substantially decreases, the symptoms associated with the diseases or conditions of the present invention.

The treatment method according to the teachings of the present invention includes implanting in a tissue region of a subject biomedical device 12 and administering (e.g., systemically) to the subject delivery vehicle 14 carrying therapeutic agent 28.

Implantation of biomedical device 12, may be effected by any implantation method known in the art, depending on the size and structure of the biomedical device as well as the tissue region to be implanted (i.e., size and anatomical and physiological state). For example, biomedical device 12 used for percutaneous transluminal angioplasty of arteriosclerotic deposits or atheroma in the carotid artery is implanted by an outermost guide catheter, which is pushed through an opening in the inguinal region of the subject into the vessel, until its front opening is situated directly in front of the stenosis. An innermost occlusion catheter is then inserted into the guide catheter and placed in a way that the occlusion balloon can be stabilized in the inflated state distal of the stenosis. A central dilation catheter is then pushed over the occlusion catheter, and the dilatation balloon is positioned in the middle of the stenosis which is now dilated in a known manner.

Delivery vehicle 14 can be administered to a subject intravenously, intramuscularly, or subcutaneously or in any manner appropriate to the therapeutic effect desired, including intranasaly (as an aerosol).

Delivery vehicle 14 can be lyophilized and then formulated into an aqueous suspension in a range of microgram/ml to 100 mg/ml prior to use.

The desired concentration of agent 28 in carrier particle 20 depends on absorption, inactivation, and excretion rates of the agent as well as the release rate of the agent from the carrier. It will be appreciated that dosage values will also vary with the severity of a condition to be alleviated. It is to be further understood that for any particular subject, specific dosage regimens should be adjusted over time according to the individual need and the professional judgment of the person administering or supervising the administration of the compositions.

One distinct advantage of system 10 of the present invention is that it enables repeated loading of a single implanted device.

Thus, delivery vehicle 14 can be administered once, or several times over a predetermined period of time. Repeated loading of biomedical device 12 (reloading) enables delivery of any number of doses (of delivery vehicle 14) over an extended time period, thus enabling long term treatment of a disorder. The number of doses loaded and the time intervals between loading can be planned for each case according to the release rate of the therapeutic agent from carrier particle 20, the dissociation rate of first member 18 from second member 22 and the desired dosage per time period.

To ensure system reloadability, the binding pair is selected, such that it dissociates following release of therapeutic agent 28. It will be appreciated that dissociation rate of the binding pair depends on the K_{off} value for the binding pair.

Examples for avidin derivatives with differential K_{off} values, which can be used with a reloadable configuration of the present invention are summarized in Table 2, below.

Table 2

| Dissociation rate and half life | PH | | | | | | |
|---|---|---|---|---|---|---|---|
| time | 1.7 | 2 | 3 | 5 | 7 | 9.2 | 10.5 |
| Avidin K_{off}(sec⁻¹ · 10⁷) | — | 200 | 9 | 0.9 | 0.4 | — | — |
| Avidin T ½ | — | 0.4 | 9 | 90 | 200 | — | — |
| Streptavidin K_{off}(sec⁻¹ · 10⁷) · | 35 | — | 19 | 8.7 | 28 | 64 | 100 |
| Streptavidin T1/2 | 2.3 | — | 4.2 | 9.2 | 2.9 | 1.25 | 0.8 |

Delivery vehicle 14 is preferably formulated for intravenous, intramuscular, or topical application, or any other suitable delivery routes.

Saturated or non-saturated avidin is preferably used as second member 22, since it has been shown that avidin shuttles improve delivery of drug derivatives through epithelial barriers, such as those encountered by oral administration [Guy M et al. (2001) Cell Physiol. Biochem. 11:271-8].

As such, delivery vehicle is preferably provided in a solution or suspension which includes a sterile diluent such as water or saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

A parental formulation can be enclosed in ampoules, disposable syringes or multiple dose vials made of glass or plastic.

Although targeted delivery systems are known in the art (see for example U.S. Pat. Nos. 6,238,872, 6,344,028) such systems suffer from several inherent limitations.

Such prior art systems require that the therapeutic agent used thereby is modified to include a device-targeting moiety. Thus, any therapeutic agent to be used with such systems must be chemically modified (e.g., biotinylated) prior to use, substantially complicating use and adaptability of such systems. In addition, since such systems do not mask the biological activity of the therapeutic agent, administration of the modified and biologically active therapeutic agent into the body (in particular systemic administration) may lead to undesirable side effects, thus requiring low dosages to be administered.

In sharp contrast, the present invention provides a biomedical system for targeted delivery of therapeutic agents which system enables drug-reloading of an implanted device without having to employ invasive procedures such as catheterization or surgery.

The biomedical system of the present invention uses carrier particles for storing the therapeutic agent delivered, thus making the present invention adaptable to carrying any drug type without having to effect costly and at times difficult drug modifications.

In addition, the novel configuration of the biomedical system of the present invention enables concomitant or separate delivery of one or more drug types while enabling to control drug release at desired target sites and in accordance with a desired treatment regimen.

Finally, the novel configuration of the biomedical system of the present invention ensures that the drug delivered does not exert its biological activity at undesired tissue locations, since vesicle packaging masks the biological activity of the delivered therapeutic agent until targeted release.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include various chemical and biochemical techniques. The procedures used herein are believed to be well known in the art and are provided for the convenience of the reader. General references describing methodology which can be utilized with the procedure used herein are provided throughout this document.

### Example 1

### Biotin Conjugation—Procedures and Reagents

Protein biotinylation—Biotinylation of bovine serum albumin (BSA) is effected according to the method of Heitzmann and Richards [Proc.Natl.Acad.Sci.U.S.A. 71,3537 (1974)].

BSA (500 mg, SIGMA, IL) is dissolved in 50 ml of ice-cold 0.1M NaHCO₃ (pH 7.5). Thereafter, Biotin N-hydroxysuccinimide ester (5 ml, BNHS, Pierce Chemical Co) at a concentration of 12 mg/ml in N,N-dimethylformamide is added to the BSA solution. The mixture is incubated overnight at 4 C with gentle stirring. Following incubation, the mixture is dialyzed against distilled and deionized water to remove unreacted BNHS and finally buffered by dialyzing against HEPES buffer. Biotin-BSA conjugates are aliquoted (200 μl/tube) and stored frozen in microcentrifuge tubes at −20° C.; the preparation is stable for one year.

Biotinylation of BSA is assessed by ¹²⁵I-avidin binding assay, which measures the amount of iodinated avidin, which is capable of binding the Biotin-BSA conjugates.

Polystyrene wells (96 wells/plate) are coated with either Biotin-BSA (prepared as above) or native BSA (dissolved in 5 mg/ml of NaHCO₃) 100 μl/well, overnight at 4° C. and thereafter unbound BSA is discarded. Coated plates are blocked with 3% BSA in PBS and washed three times with 200 μl of 0.05% Tween-20 in PBS. Iodination of avidin is effected using the Bolton-Hunter procedure [Guy et al. (2001) Cell Physiol. Biochem. 11:271-278]. Serial dilutions of radio-labeled avidin are then incubated with biotin-BSA coated wells overnight at 4° C. Following incubation, wells are washed, separated, and counted. Biotin-BSA coated wells bind 50-100 times more ¹²⁵I-labeled avidin than native BSA coated wells.

Colloidal gold biotinylation—Pharmaceutically acceptable colloidal gold nanoparticals are preferred for in-vivo application. Monoamino Nanogold (Nanoprobes-2021) or positively charged Nanogold (Nanoprobes-2022) particles are reacted with LC-NHS-biotin (Pierce).

Nanogold reagent is resuspended in 0.2 ml of DMSO or isopropanol and 0.8 ml of buffer (0.02 M HEPES-sodium hydroxide at pH 8.2). Nanogold solution is then incubated with 10 to 20-fold excess LC-NHS-biortin in the same buffer for 1 hour at room temperature and overnight agitation at 4° C. Conjugates are separated by gel filtration [GH25, Millipore, or Superdex-Peptide (Pharmacia)], which fractionates Biotinylated Nanogold, free Nanogold and small molecules such as biotin.

FITC-labled Colloidal gold biotinylation—Fluorescein (FITC)-Conjugated gold particles (Nanogold, Nanoprobes, Inc. Stony Brook, N.Y., 250 nmol) are lyophilized with methanol. Dried FITC-Conjugated gold particles are then dissolved in DMSO (0.4 ml) and 0.02M HEPES-NaOH buffer, pH 7.5 (0.9 ml). The mixture is added to a cross-linking solution of bis (sulfo-N-hydroxysuccinimidyl) suberate (BS₃) (250-fold excess: 38 mg, Pierce Chemical Co.) dissolved in DMSO (0.1 M). The solution is mixed thoroughly and incubated at room temperature for 1 hour and 25 minutes. Thereafter, sulfo-NHS-Fluorescein-Nanogold is separated from excess cross-linker (BS₃) by chromatography on a coarse gel filtration column (GH25, Amicon: length=50 cm, internal diameter=1.0 cm, volume=40 ml), and elution with 0.02M HEPES-NaOH, pH 7.5, in 20% isopropanol/water.

Activated fluorescein-Nanogold is then allowed to react with 12-fold excess biotin in an aqueous solution of the same buffer overnight at 4° C. Reaction volume is then reduced to 0.5 ml by membrane centrifugation (Centricon-30, Amicon) and purified twice by gel filtration (Amicon GCL-90 gel: Column length=50 cm, internal diameter=0.66 cm, volume=16 ml). Elution is effected with 0.02 M sodium phosphate buffer, pH 7.4 with 150 Mm NaCl.

Gold dendromers biotinylation—is described by Hyun C. Yoon et al. 2000 Analytical Biochemistry 282, 121-128.

Albumin-Biotin-colloidal gold conjugates—Particles of 5 nm and 20 nm in diameter are available from Sigma-Aldrich (#A5547 or A4417).

### Example 2

### Loading of Fluorescein-Labled Avidin Derivatives onto a Biotin-Coated Stent Model

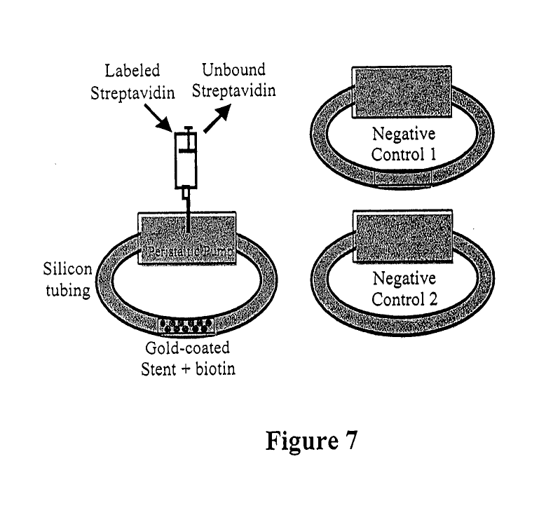

An Outline of a stent loading procedure is schematically illustrated in FIG. 7.

A Biotin conjugated gold-coated stent is placed in a silicon tubing, which is joined in a close loop to a peristaltic pump [Monnink et al. (1999) J. Investig. Med. 47:304-310].

Blood samples obtained from healthy and drug-free volunteers and retrieved (6 ml) from antecubital veins using 16 G needles. The blood samples are collected into heparanized plastic syringes, which are placed into the silicon tubing. Blood is circulated at 8 ml/minute at 37° C.

FITC conjugated streptavidin (Pierce Chemical Co. Cat No.: 21224ZZ) or Neutravidin (Pierce Chemical Co. Cat No.: 31006ZZ) is injected into the loop, such that a portion of avidin conjugates are bound to the biotin coated stent, while the remaining non-bound avidin derivatives continue to circulate within the close loop. Various concentrations of FITC-conjugated avidin derivatives are applied in-order to determine the binding capacity of the biotin-coated stent. Furthermore, a system consisting of a lose loop but lacking a biotin coated stent, and a system consisting of a lose loop and non-biotin coated stent comprise negative controls.

Following binding equilibration, in which various time frames are tested (i.e., 0.5, 1, 2, 5, 10 minutes), total volume of circulating blood is collected.

To determine binding capacity and specificity, serum fraction is separated from whole blood by centrifugation. The amount unbound labeled avidin derivative is determined by a standard fluorometric assay [Mock and Horowitz (1990) Meth. Enzymol. Vol. 184, Eds. Wilchek and Bayer Academic Press, San Diego 234-240; Haugland (1996) Handbook of fluorescent probes and research chemicals 6^{th} Ed. 137-141].

Other labeling methods may be used to determine binding capacity. For example, radiolabeled-avidin derivatives can be used, in which case evaluation is effected by a radioassay.

### Example 3

### FPLC Determination of Fluorescent-Avidin Binding to Biotin Coated Beads

The influence of various parameters such as flow rate, temperature and pH on fluorescent-avidin-derivatives binding to biotin coated beads is determined by FPLC coupled to fluorometric detection.

### Example 4

### Biotin Distribution on a Stent Device

The dose distribution of radiolabled biotin (³²P) coated stents is calculated in water surrounding the stents, according to the convolution method and measured by exposing radiochromic film in a solid-water phantom [Dugan et al. (1998) Int. J. Radiation Oncology Biol. Phys. 40:713-720].

### Example 5

### Animal Models of Malignant Diseases and In-Restenosis for Studying the Biomedical Systems of the Present Invention

In stent restenosis—(i) The swine/microswine models of iliac or coronary neointimal proliferation are taught by Laird et al. (1996) Circulation 93: 529-536 (swine iliac); Fischell et al. (1996) Am. J. Cardiol. 78 (suppl. 3A): 45-50 (swine coronary); Lincoff et al. (1997) J. Am. Coll. Cardiol. 29: 808-816 (swine coronary) and Fontaine and Dos Passos (1997) J. Vasc. Interv. Radiol. 8: 107-111 (microswine). (ii) The rabbit iliac artery injury model is taught by Carter et al. (1999) Cardiovasc. Pathol. 8: 73-80 and Foo et al. (2000) Thromb. Haemost. 83: 496-502.

Cancer—Nanogold beads can be tested in cancer models i.e., breast cancer and intestinal adenoma provided by Misdorp W and Weijer K. Animal Model of human disease: breast cancer. Am. J Pathol. (1980) 98 (2): 573-6 and Moser A R et al. ApcMIn: a moiuse model for intestinal mammary tumorogenesis.(1995) Eur J Cancer 31 A (7-8):1061-4.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent, or patent application was specifically and individually indicated to be incorporated herein by reference. In addition, citation or identification of any reference in this application shall not be construed as an admission that such reference is available as prior art to the present invention.

What is claimed is:

1. A biomedical system for targeted delivery of a therapeutic agent to a tissue region of a subject, the biomedical system comprising:
(a) a biomedical device including:
(i) a device body designed and configured for implantation within the tissue region of the subject; and
(ii) a first member of a binding pair attached to a surface of said device body; and
(b) a delivery vehicle including:
(i) a carrier particle designed for carrying the therapeutic agent;
(ii) a second member of said binding pair attached to said carrier particle, said second member of said binding pair being capable of specifically interacting with said first member of said binding pair thereby enabling targeting of said delivery vehicle to said biomedical device when implanted within said tissue region.

2. The biomedical system of claim 1, wherein said delivery vehicle is configured such that when said first member and said second member of said binding pair interact, the therapeutic agent carried by said delivery vehicle is released therefrom.

3. The biomedical system of claim 1, wherein said biomedical device further includes an effector moiety attached to said surface of said device body, said effector moiety being designed for activating release of the therapeutic agent carried by said delivery vehicle when said first member and said second member of said binding pair interact.

4. The biomedical system of claim 3, wherein said effector moiety forms a part of said first member of said binding pair.

5. The biomedical system of claim 3, wherein said effector moiety is a chemical selected from the group consisting of a nonionic chemical, an anionic chemical, a cationic chemical, a natural occurring chemical and an amphoteric chemical

6. The biomedical system of claim 3, wherein said effector moiety is an enzyme selected from the group consisting of a lipase and a peptidase.

7. The biomedical system of claim 1, wherein said biomedical device is a stent.

8. The biomedical system of claim 1, wherein said binding pair is selected from the group consisting of a biotin-avidin pair, a hapten-antigen pair, a lectin-carbohydrate pair and a ligand-receptor pair.

9. The biomedical system of claim 1, wherein said carrier particle is selected from the group consisting of a liposome, a micelle, a cationic polymer and a cationic peptide.

10. A biomedical system for repeated targeting of a therapeutic agent to a tissue region of a subject, the biomedical system comprising:
(a) a biomedical device including:
(i) a device body designed and configured for implantation within the tissue region of the subject; and
(ii) a first member of a binding pair attached to a surface of said device body;
(b) a delivery vehicle including:
(i) a carrier particle designed for carrying the therapeutic agent;
(ii) a second member of said binding pair attached to a surface of said carrier particle, said second member of said binding pair being capable of specifically interacting with said first member of said binding pair;
wherein said first and second members of said binding pair and said delivery vehicle are selected such that following targeting, the therapeutic agent is released from said delivery vehicle and said first member dissociates from said second member of said binding pair, thereby enabling repeated targeting of the therapeutic agent to the tissue region of the subject.

11. The biomedical system of claim 10, wherein said release of the therapeutic agent from said delivery vehicle is effected by an effector moiety attached to said surface of said device body.

12. The biomedical system of claim 11, wherein said effector moiety is designed for activating release of the therapeutic agent from said delivery vehicle upon interaction between said first and said second members of said binding pair.

13. The biomedical system of claim 10, wherein said delivery vehicle is configured such that when said first member and said second member of said binding pair interact, the therapeutic agent carried by said delivery vehicle is released therefrom.

14. The biomedical system of claim 11, wherein said effector moiety forms a part of said first member of said binding pair.

15. The biomedical system of claim 11, wherein said effector moiety is a chemical selected from the group consisting of a nonionic chemical, an anionic chemical, a cationic chemical, a natural occurring chemical and an amphoteric chemical

16. The biomedical system of claim 11, wherein said effector moiety is an enzyme selected from the group consisting of a lipase and a peptidase.

17. The biomedical system of claim 11, wherein said biomedical device is a stent.

18. The biomedical system of claim 10, wherein said binding pair is selected from the group consisting of a biotin-avidin pair, a hapten-antigen pair, a lectin-carbohydrate pair and a ligand-receptor pair.

19. The biomedical system of claim 10, wherein said carrier particle is selected from the group consisting of a liposome, a micelle, a cationic polymer and a cationic peptide.

20. A drug-reloadable biomedical implant comprising:
(a) a device body designed and configured for implantation within a tissue region of a subject;
(b) a first member of a binding pair attached to a surface of said device body, said first member of said binding pair being capable of interacting with a second member of said binding pair attached to a delivery vehicle carrying a therapeutic agent, thereby enabling targeting of said delivery vehicle to the biological implant; and
(c) an effector moiety attached to said surface of said device body, said effector moiety being designed for activating release of a therapeutic agent from said delivery vehicle upon interaction between said first and said second members of said binding pair.

21. The biomedical system of claim 20, wherein said effector moiety forms a part of said first member of said binding pair.

22. The biomedical system of claim 20, wherein said effector moiety is a chemical selected from the group consisting of a nonionic chemical, an anionic chemical, a cationic chemical, a natural occurring chemical and an amphoteric chemical

23. The biomedical system of claim 20, wherein said effector moiety is an enzyme selected from the group consisting of a lipase and a peptidase.

24. The biomedical system of claim 20, wherein the biomedical implant is a stent.

25. The biomedical system of claim 20, wherein said binding pair is selected from the group consisting of a biotin-avidin pair, a hapten-antigen pair, a lectin-carbohydrate pair and a ligand-receptor pair.

26. A method of delivering a therapeutic agent to a tissue region of a subject, the method comprising:
(a) implanting in the tissue region of the subject a biomedical device including:
(i) a device body; and
(ii) a first member of a binding pair attached to a surface of said device body;
(b) administering to the subject a delivery vehicle carrying the therapeutic agent, the delivery vehicle including:
(i) a carrier particle designed for carrying the therapeutic agent;
(ii) a second member of said binding pair attached to said carrier particle, said second member of said binding pair being capable of specifically interacting with said first member of said binding pair thereby enabling targeting of said delivery vehicle to said biomedical device and said tissue region.

27. The method of claim 26, wherein said biomedical device further includes an effector moiety attached to said surface of said device body, said effector moiety being designed for effecting release of said therapeutic agent carried by said delivery vehicle when said first member and said second member of said binding pair interact.

28. The method of claim 27, wherein said effector moiety forms a part of said first member of said binding pair.

29. The method of claim 27, wherein said effector moiety is a chemical selected from the group consisting of a nonionic chemical, an anionic chemical, a cationic chemical, a natural occurring chemical and an amphoteric chemical

30. The method of claim 27, wherein said effector moiety is an enzyme selected from the group consisting of a lipase and a peptidase.

31. The method of claim 26, wherein said biomedical device is a stent.

32. The method of claim 26, wherein said binding pair is selected from the group consisting of a biotin-avidin pair, a hapten-antigen pair, a lectin-carbohydrate pair and a ligand-receptor pair.

33. The method of claim 26, wherein said carrier particle is selected from the group consisting of a liposome, a micelle, a cationic polymer and a cationic peptide.

34. The method of claim 26, wherein said first and second members of said binding pair dissociate following release of the therapeutic agent, thereby enabling repeating step (b).

35. The method of claim 34, further comprising repeating step (b) a predetermined number of times.

36. A method of manufacturing a reloadable biomedical device the method comprising:
(a) fabricating a device body designed and configured for implantation within a tissue region of a subject;
(b) attaching to a surface of said device body a first member of a binding pair, said first member of said binding pair being capable of interacting with a second member of said binding pair attached to a delivery vehicle carrying a therapeutic agent, thereby enabling targeting of said delivery vehicle to the biological implant; and
(c) attaching to said surface of said device body an effector moiety said effector moiety being designed for effecting release of said therapeutic agent from said delivery vehicle upon interaction between said first and said second members of said binding pair.

37. The method of claim 36, wherein said effector moiety forms a part of first member of said binding pair.

38. The method of claim 36, wherein said effector moiety is a chemical selected from the group consisting of a nonionic chemical, an anionic chemical, a cationic chemical, a natural occurring chemical and an amphoteric chemical

39. The method of claim 36, wherein said effector moiety is an enzyme selected from the group consisting of a lipase and a peptidase.

40. The method of claim 36, wherein said binding pair is selected from the group consisting of a biotin-avidin pair, a hapten-antigen pair, a lectin-carbohydrate pair and a ligand-receptor pair.
